(19) United States
(12) Patent Application Publication
(10) Pub. No.: US 2007/0272240 A1
(43) Pub. Date: Nov. 29, 2007

(54) HOSE SYSTEM FOR BILATERAL POSITIVE AIRWAY PRESSURE DEVICE

(75) Inventors: Alonzo C. Aylsworth, Wildwood, MO (US); Charles R. Aylsworth, Wildwood, MO (US)

Correspondence Address: CONLEY ROSE, P.C.;David A. Rose, P. O. BOX 3267, HOUSTON, TX, 77253-3267, US

(73) Assignee: ACOBA, LLC., Chesterfield, MO (US)

(21) Appl. No.: 11/381,612

(22) Filed: May 4, 2006

(51) Int. Cl. A61M 16/00

(52) U.S. Cl. 128/204.18

(57) ABSTRACT

At least some of the illustrative embodiments are hose systems for use with a positive airway pressure device comprising a first hose configured to fluidly couple on a device end to a first outlet of the positive airway pressure device and on a patient end to a first naris of a patient, and a second hose configured to fluidly couple on a device end to a second outlet of the positive airway pressure device and on a patient end to a second naris of the patient (wherein the second hose is fluidly independent from the first hose). The hose system is configured such that the first hose only couples to the first outlet and the second hose only couples to the second outlet.

### BACKGROUND

Sleep disordered breathing is common throughout the population. Some sleep disorders may be attributable to disorders of the respiratory tract. Sleep apnea may be a disorder where a person temporarily stops breathing during sleep. A hypopnea may be a period of time where a person's breathing becomes abnormally slow or shallow. In some cases, a hypopnea precedes an apnea event.

Although hypopneas and apneas may have multiple causes, one trigger for these type events may be full or partial blockages in the respiratory tract. In particular, in some patients the larynx may collapse due to forces of gravity and/or due to forces associated with lower pressure in the larynx than outside the body. A collapse of the pharynx, larynx, upper airway or other soft tissue in the respiratory tract may thus cause a full or partial blockage, which may lead to a hypopnea or apnea event.

One method to counter collapse of the larynx may be the application of positive airway pressure, possibly by using a continuous positive airway pressure (CPAP) machine. This may be accomplished in the related art by placing a mask over at least the patient's nose, and providing within the mask a pressure communicated to the pharynx, larynx, or upper airway. The pressure within the pharynx, larynx, or upper airway may be greater than the pressure outside the body, thus splinting the airway open.

### SUMMARY

There are therapeutic advantages to a positive airway pressure device individually applying and controlling pressure to each naris of the patient, and such a device may thus be called a bilateral positive airway pressure device. As an example of a therapeutic advantage, where one naris has greater resistance to airflow, the pressure applied to that particular naris may be increased such that airflow is more evenly distributed as between the nares. A positive airway pressure device may have the ability to log pressure applied to each naris, and the log may be helpful in diagnosing certain ailments (e.g., existence of a tumor or polyp in a particular naris, head position dependent valve collapse in a particular naris). For the log to be most beneficial in diagnosis, the output port that the bilateral positive airway pressure device expects to be coupled to the right naris should be coupled to the right naris, and the output port that the bilateral positive airway pressure device expects to be coupled to the left naris should be coupled to the left naris. Moreover, each nasal mask for positive airway pressure applications has a controlled leak to allow escape of exhaled carbon dioxide, and the controlled leak rate may be different on each style of mask. Knowing the controlled leak rate may be helpful to the bilateral positive airway pressure device in determining whether there is a nasal mask seal leak. Thus, ensuring proper connection of hoses to the bilateral positive airway pressure device and/or identifying the type mask used by the patient would be helpful to address these concerns.

To that end, at least some of the illustrative embodiments are systems comprising a positive airway pressure device and a hose system. The positive airway pressure device comprises a first outlet port that provides positive airway pressure to a first naris of a patient and a second outlet port that provides positive airway pressure to a second naris of a patient. The positive airway pressure supplied to each naris is individually controlled. The hose system comprises a first hose configured to fluidly couple on a device end to the first outlet port (and the first hose configured to fluidly couple between the first outlet port and the first naris of the patient) and a second hose configured to fluidly couple on a device end to the second outlet port (and the second hose configured to fluidly couple between the second outlet port and the second nauis of the patient). The hose system is configured such that the first hose only couples to the first outlet port and the second hose only couples to the second outlet port.

Other illustrative embodiments are hose systems for use with a positive airway pressure device comprising a first hose configured to fluidly couple on a device end to a first outlet of a positive airway pressure device and on a patient end to a first naris of a patient, and a second hose configured to fluidly couple on a device end to a second outlet of a positive airway pressure device and on a patient end to a second naris of the patient (wherein the second hose is fluidly independent from the first hose). The hose system is configured such that the first hose only couples to the first outlet and the second hose only couples to the second outlet.

The disclosed devices and methods comprise a combination of features and advantages which enable it to overcome the deficiencies of the prior art devices. The various characteristics described above, as well as other features, will be readily apparent to those skilled in the art upon reading the following detailed description, and by referring to the accompanying drawings.

### BRIEF DESCRIPTION OF THE DRAWINGS

For a detailed description of the preferred embodiments of the invention, reference will now be made to the accompanying drawings in which:

FIG. 1 illustrates a system in accordance with embodiments of the invention;

FIG. 2 illustrates hose connections in accordance with some embodiments;

FIG. 3 illustrates hose connections in accordance with some embodiments;

FIG. 4 illustrates hose connections in accordance with some embodiments;

FIG. 5 illustrates hose connections that also identify the mask, in accordance with some embodiments; and

FIG. 6 illustrates an electrical circuit in accordance with some embodiments.

### NOTATION AND NOMENCLATURE

Certain terms are used throughout the following description and claims to refer to particular system components. This document does not intend to distinguish between components that differ in name but not function.

In the following discussion and in the claims, the terms “including” and “comprising” are used in an open-ended fashion, and thus should be interpreted to mean “including, but not limited to . . . ”. Also, the term “couple” or “couples” is intended to mean either an indirect or direct connection. Thus, if a first device couples to a second device, that connection may be through a direct connection, or through an indirect connection via other devices and connections.

### DETAILED DESCRIPTION

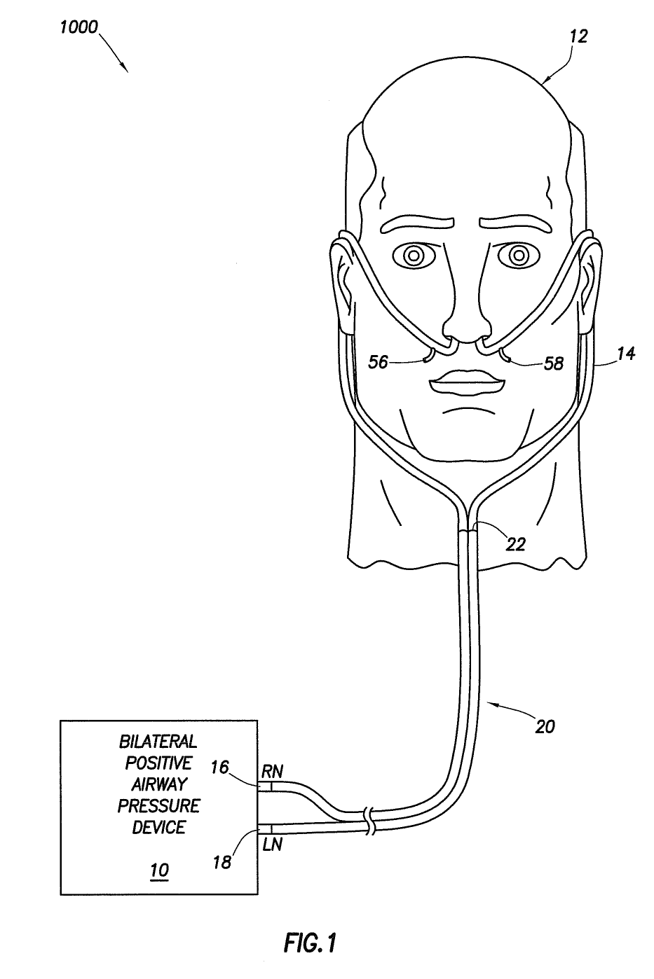

FIG. 1 illustrates a system 1000 in accordance with embodiments of the invention. In particular, FIG. 1 illustrates a bilateral positive airway pressure device 10. A bilateral positive air pressure device such as illustrated in FIG. 1 is a device that applies positive airway pressure to a patient's nares on an individual basis. In this way, each naris is supplied a pressure best suited for assisting the patient's breathing through that naris. The bilateral positive airway pressure device 10 may be a device such as described in co-pending patent application Ser. No. 10/851,952, titled “Method and System of Individually Controlling Airway Pressure of a Patient's Nares,” assigned to the same entity as the current specification, and incorporated by reference herein as if reproduced in full below.

The individually controlled pressures are coupled to a patient's nares by way of a nasal mask. For example, FIG. 1 illustrates a patient 12 wearing a mask 14 with two fluidly independent pathways. The right nails fluidly couples to a right nalis port 16 and the left naris fluidly couples to a left nalis port 18. In some embodiments, the hose system 20, that fluidly couples the bilateral positive airway pressure device 10 to the mask 14, is integral with the mask 14. In alternative embodiments, the hose system 20 fluidly couples on a device end to the outlet ports 16 and 18, and fluidly couples on a patient end to the nasal mask 14, such as at connection location 22.

Still referring to FIG. 1, a positive airway pressure device 10 in accordance with at least some embodiments of the invention has the ability to not only apply positive airway pressure to the patient's nares on an individual basis, but also to log information about the applied pressures. The information regarding the amount of pressure used to equalize or substantially equalize the airflow as between the patient's nares may be helpful in diagnosing certain ailments (e.g., existence of a tumor or polyp in a particular naris, head position dependent of valve collapse in a particular naris). For the log to be most beneficial in diagnosis, the output port 16 for the right naris should be coupled to the right naris, and the output port 18 for the left naris should be coupled to the left naris. In accordance with at least some embodiments of the invention, the positive airway pressure device 10 and the hose system 20 work together to ensure that the hose for the right naris only couples to the outlet port 16 for the right naris, and the hose for the left naris only couples to the outlet port 18 for the left nalis.

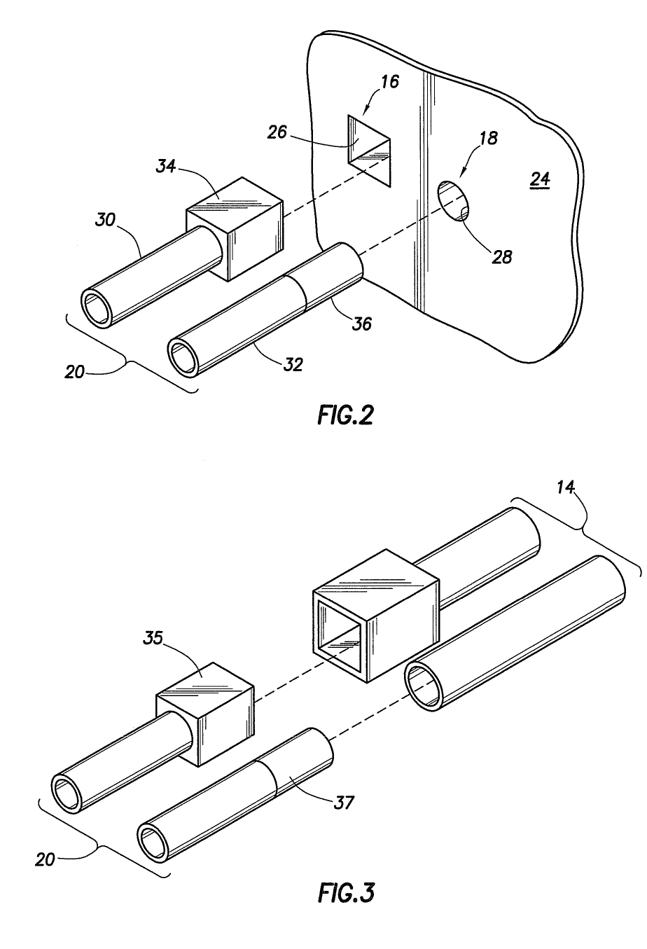

Ensuring the proper coupling of the hose system 20 and mask 14 to the bilateral positive airway pressure device 10 may talke many forms. In some embodiments, each hose has a device-end fitting that mates only with the appropriate outlet port. FIG. 2 illustrates a system where the device-end fittings ensure proper orientation. In particular, FIG. 2 illustrates a partial view of an exterior 24 of a positive airway pressure device. The illustrative exterior has two outlet ports 16 and 18, which in these embodiments comprise apertures 26 and 28. The apertures 26 and 28 fluidly couple to fans and/or blowers which provide the positive airway pressure to the patient. FIG. 2 also illustrates a portion of the hose system 20 comprising a first hose 30 and a second hose 32. Each of the hoses 30 and 32 have a device-end fitting 34 and 36 respectively. As illustrated in FIG. 2, the device-end fitting 34 is configured to fluidly couple to the aperture 26, but because of the difference in aperture shape the device-end fitting 34 will not couple to the aperture 28. Likewise the device-end fitting 36 is configured to fluidly couple to the aperture 28, but because of the difference aperture shape the device-end fitting 36 will not fluidly couple the aperture 26. In this way, the hose system 20 cannot be fluidly coupled to the positive airway pressure device 10 in a reverse order. While FIG. 2 shows an illustrative square aperture 26 and circular aperture 28 (and corresponding device-end fittings 34 and 36), other shapes and configurations may be equivalently used. For example, the bilateral positive airway pressure device 10 may have one male and one female connection, and likewise the hose system 20 may have one male and one female connection.

In embodiments where the mask 14 is integral with the hose system 20, the connection to the bilateral positive airway pressure device 10 completes the fluid circuit. However, in alternative embodiments, the mask 14 may be a separate component from the hose system 20, and in these embodiments the hose system 20 fluidly couples to the mask 14, for example at connection location 22. For the same reasons as the discussed above, it is desirable to ensure that at the connection location 22 the fluid connection for the right naris continues to the right naris, and the fluid connection for the left naris continues to the left naris. In these embodiments, the patient end of the hose system 20 may be configured such that it couples to the nasal mask 14 in only one orientation. FIG. 3 illustrates embodiments where the hose system 20 patient-end fitting 35 is configured to couple to only one of the hoses for the mask 14. Likewise, the patient-end fitting 37 of the hose system 20 is configured to couple to only one of the hoses of the nasal mask 14. While FIGS. 2 and 3 illustrate rectangular and circular connections, any structural differences between the two hose connections that eliminates the possibility of misconnecting hoses may be equivalently used.

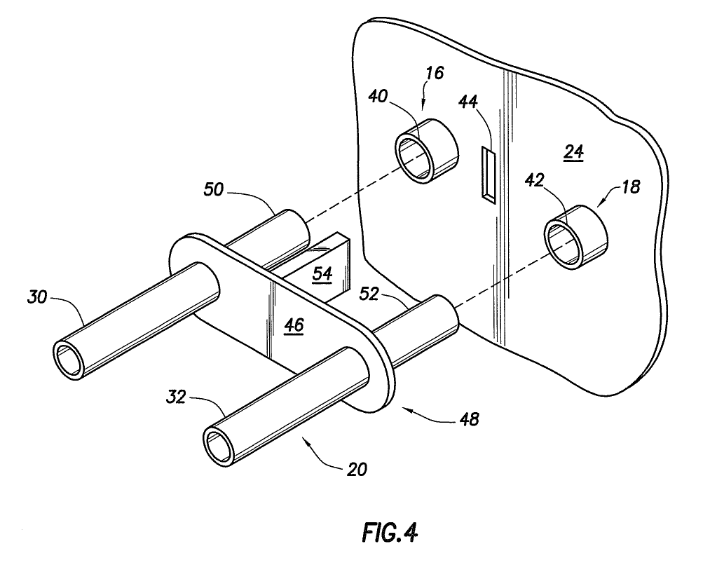

FIG. 4 illustrates alternative embodiments that ensure proper coupling of the hose system 20 to the bilateral positive airway pressure device 10. In particular, FIG. 4 illustrates the partial view of the exterior 24 of the positive airway pressure device. The illustrative exterior has two outlet ports 16 and 18, which in these embodiments comprises male connections 40 and 42 respectively. Female connections may be equivalently used. The illustrative exterior 24 also has a key aperture 44. The connections 40 and 42 couple one each to fans and/or blowers which provide the positive airway pressure to the patient. FIG. 4 also illustrates a portion of the hose system 20 comprising a first hose 30 and second hose 32. A bracket 46 mechanically (though not fluidly) couples the hoses 30 and 32 together on the device end 48. Each of the hoses 30 and 32 has a device-end fitting 50 and 52, respectively; however, the device-end fittings 50 and 52 may be similar in these embodiments because the bracket 46 and a tab 54 work together with the aperture 44 in the bilateral positive airway pressure device to ensure that the house is coupled in only one orientation.

As illustrated in FIG. 4, the key aperture 44 is positioned off center with respect to the outlet ports 16 and 18. Likewise, the tab 54 coupled to the bracket 46 is positioned off center with respect to the bracket 46. In this way, the device end 48 of the hose system 20 couples the hoses 30 and 32 to their respective outlet ports 16 and 18 in only one orientation - the orientation where the tab 54 extends into the key aperture 44. If a user attempts to fluidly couple the hoses 30 and 32 to the bilateral positive airway pressure device in a reverse orientation, the key aperture 44 will not align with the tab 54, and thus the hose system 20 will not fluidly couple to the bilateral positive airway pressure device 10. The long dimension of the key aperture 44, and correspondingly the long dimension of the tab 54, may be equivalently oriented at any angle. In alternative embodiments, the key aperture 44 and the tab 54 may equivalently use other corresponding shapes (e.g. circular, square, hexagonal), so long as the key aperture 44 and tab 54 allow the hose system 20 to fluidly couple to hoses 30 and 32 to the ports 16 and 18 in only one orientation. Further still, the key aperture 44 and tab 54 need not be disposed between the outlet ports 16 and 18, and thus the key aperture 44 and the tab 54 may be equivalently above or below the outlet ports 16 and 18 so long as the hose system 20 couples to the outlet ports 16 and 18 in only one orientation.

Returning now to FIG. 1, nasal mask 14 is merely illustrative of nasal masks that may be used with a bilateral positive airway pressure device 10. While the nasal mask 14 is shown to insert within each nostril and therefore to seal to the internal diameter of the nostrils, other nasal masks may use other coupling methodologies, such as nasal pillows. Regardless of the system used, however, it is not uncommon for there to be leaks at the interface between the hose portion and the naris. Moreover, nasal masks also have vent ports (e.g. vent ports 56 and 58) that allow carbon dioxide to escape during the exhalation portion of the respiration, but also allow airflow from the bilateral positive airway pressure device to escape during the inhalation portion of the respiration. In spite of having ports 56 and 58 to allow escape of carbon dioxide, bilateral positive airway pressure devices 10 in accordance with some embodiments implement a software-based method to detect when the leaks at the interface between the hoses and the patient's naris become excessive. In order to accomplish this task, however, the bilateral positive airway pressure device 10 takes into account the amount of air escaping through the vents 56 and 58. The amount of air escaping from the vents 56 and 58 varies based on the manufacturer and type of mask.

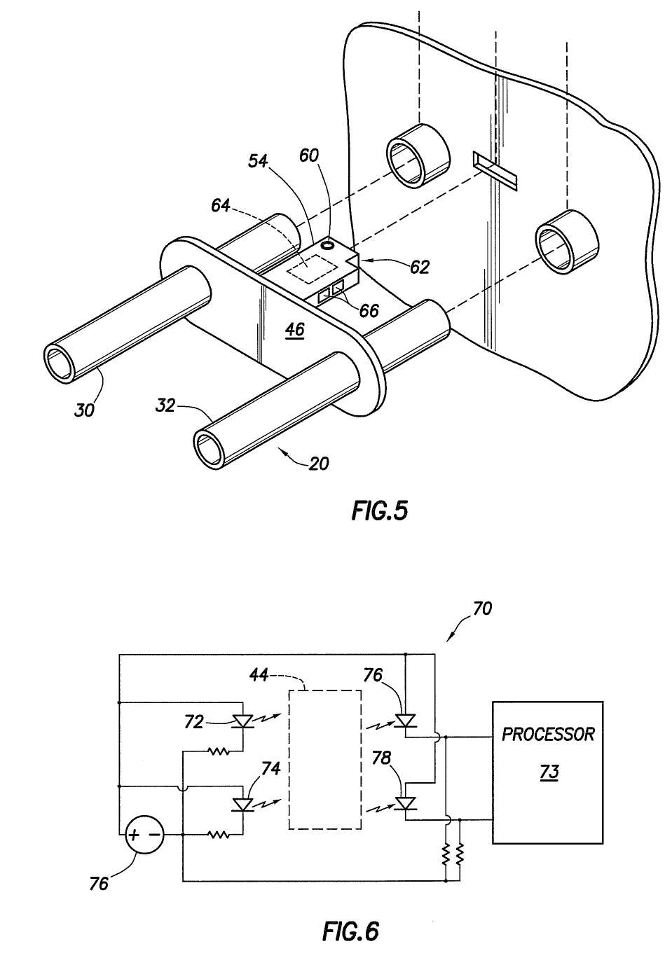

Because of the varying controlled leaks, and in accordance with some embodiments of the invention, the bilateral positive airway pressure device 10 works in combination with the hose system 20 to identify the mask 14 used by the patient 12. FIG. 5 illustrates embodiments where the hose system 20 identifies the mask used by the patient. In particular, the tab 54 of the bracket 46 has features that identify the nasal mask to which the hoses 30 and 32 couple. The term “features” in this specification and in the claims is used broadly to encompass not only physical features (e.g., aperture 60 or notch 62), but the term features also comprises any mechanism affiliated or associated with the tab 54 that identifies the nasal mask (e.g., embedded electronic device 64). The embedded electronic device 64 in some embodiments is a serial read only memory (ROM) which electronically couples to and communicates with a processor of the bilateral positive airway pressure device by way of electrical contacts 66. In alternative embodiments, the embedded electrical device 64 is a radio frequency identification (RFID) tag which is read by the bilateral positive airway pressure device. In yet further alternative embodiments, the tab 54 has identifying indicia on its outer surface, such as a color coding scheme or bar code that is read by the processor of the bilateral positive airway pressure device 10. Notice also that in addition to supporting the features which identify the mask, the tab 54 can also serve the purpose of ensuring that the hose system couples to the bilateral positive airway pressure device in only one orientation, as discussed above.

FIG. 6 illustrates a circuit 70 which couples to an illustrative processor 73. The processor 73 controls, in whole or in part, operation of the bilateral positive airway pressure device 10. The circuit 70 illustrated in FIG. 6 is enabled for use with features of the tab 54 being aperture 60 and/or notch 62. In particular, the circuit 70 comprises light emitting diodes 72 and 74. Electrical current supplied from source 76 flows through the diodes creating light (not necessarily visible). The light from the light emitting diode 72 and 74 is configured to shine across the aperture, the aperture illustrated by dashed line 44. The circuit 70 further comprises photo diodes 76 and 78. Photo diodes 76 and 78 are arranged to be in operational relationship with the light emitting diode 72 and 74 respectively. While the illustrative circuit of FIG. 6 shows only two light detection paths, any number of light emitting diodes and photo diodes implementing any number of light detection paths may be equivalently used. As the tab 54 is inserted through the key aperture 44, the light path between the corresponding light emitting diode and photo diode is selectively broken, and the type of mask to which the hose system 20 is attached may be identified by the pattern of broken and unbroken lights paths. For example, if the light path between the light emitting diode 72 and photo diode 76 is broken, the photo diode 76 ceases conducting, and therefore the processor 73 sees a low voltage or logic zero input. Likewise, if the tab 54 has an aperture 60 or notch 62 such that the light path is unbroken in spite of the presence of the tab 54, the photo diode conducts and the processor 73 sees a high voltage or a logic one. In the illustrative case of FIG. 6 having two light paths, three different masks may be identified (assuming that two unbroken light paths indicate that no hose system has been connected to the bilateral positive airway pressure device).

In some implementations, a patient may use a humidifier between the bilateral positive airway pressure device 10 and the hose connection. In these embodiments the humidifier (in the case of the bilateral positive airway pressure device a dual chamber humidifier) has a hose connection and an aperture system similar to those illustrated in FIGS. 4 and 5. In order for the bilateral positive airway pressure device 10 to detect the type of mask to which the hose system 20 connects, the humidifier has a tab 54 that includes fiber optics or light pipes which transfer the light from the illustrative light emitting diodes 72 and 74 to the tab of the hose system 20 coupled to the outlet ports of the humidifier. In embodiments where an electronic device such as a serial ROM is used, the humidifier has electrical contacts and corresponding electrical wiring, so that the processor 73 can electrically communicate with the serial ROM in the tab 54 coupled to the bracket 46. In embodiments where the tab 54 contains a radio frequency ID tag, the proximity of the tab 54 of the hose system 20 when fluidly coupled to the humidifier is sufficient for the tag reader to read the tag in spite of the fact that the tag may not be coupled through the aperture 44 in the bilateral positive airway pressure device.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A system comprising:
a positive airway pressure device comprising:
a first outlet port that provides positive airway pressure to a first naris of a patient; and
a second outlet port that provides positive airway pressure to a second naris of a patient;
wherein the positive airway pressure supplied to each naris is individually controlled;
a hose system comprising:
a first hose configured to fluidly couple on a device end to the first outlet port, and the first hose configured to fluidly couple between the first outlet port and the first naris of the patient;
a second hose configured to fluidly couple on a device end to the second outlet port, and the second hose configured to fluidly couple between the second outlet port and the second naris of the patient;
wherein the hose system is configured such that the first hose only couples to the first outlet port and the second hose only couples to the second outlet port.

2. The system as defined in claim 1 further comprising:
wherein the first hose has a device-end fitting that mates only with the first outlet port; and
wherein the second hose has a device-end fitting that mates only with the second outlet port.

3. The system as defined in claim 1 further comprising:
a key aperture disposed between the first outlet port and second outlet port of the positive ail-way pressure device; and
a bracket configured to hold the device ends of the first and second hoses in a fixed relationship, the bracket comprising a tab extending from the bracket substantially in the direction of the device ends of the hoses;
wherein the key aperture and the tab of the bracket are configured such that the tab extends into the aperture when the hoses are fluidly coupled to the outlet ports; and
wherein the key aperture and the tab of the bracket allow fluid coupling of the hoses to the outlet ports in only one orientation.

4. The system as defined in claim 1 further comprising:
a key aperture disposed between the first outlet port and second outlet port of the positive airway pressure device; and
a bracket configured to hold the device ends of the first and second hoses in a fixed relationship, the bracket comprising a tab extending from the bracket substantially in the direction of the device ends of the hoses, and the tab having features that identify the nasal mask to which the hoses are configured to attach;
wherein the key aperture and the tab of the bracket are configured such that the tab extends into the aperture when the hoses are fluidly coupled to the outlet ports; and
wherein the positive airway pressure device is configured to read the features of the tab that identify the nasal mask to which the noses are configured to attach.

5. The system as defined in claim 4 wherein the key aperture and the tab of the bracket enable fluid coupling of the hoses to the outlets in only one orientation.

6. The system as defined in claim 4 wherein the positive airway pressure device farther comprises:
a source of light proximate to the aperture; and
a light detector proximate to the aperture and in operational relationship to the source of light;
wherein the positive airway pressure device is configured to read the features of the tab by determining whether the tab allows light from the source of light to reach the light detector.

7. The system as defined in claim 6 further comprising:
wherein the source of light further comprises a light emitting diode; and
wherein the light detector further comprises a photo diode.

8. The system as defined in claim 4 wherein the positive airway pressure device further comprises:
a plurality of sources of light proximate to the aperture; and
a plurality of light detectors proximate to the aperture and in operational relationship one each to each source of light;
wherein the positive airway pressure device is configured to read features of the tab by determining whether the tab allows light from each source of light to reach each respective light detector.

9. A hose system for use with a positive airway pressure device, the hose system comprising:
a first hose configured to fluidly couple on a device end to a first outlet of a positive airway pressure device and on a patient end to a first naris of a patient; and
a second hose configured to fluidly couple on a device end to a second outlet of the positive airway pressure device and on a patient end to a second naris of the patient, wherein the second hose is fluidly independent from the first hose;
wherein the hose system is configured such that the first hose only couples to the first outlet and the second hose only couples to the second outlet.

10. The system as defined in claim 9 further comprising:
a first device-end fitting on the first hose that mates only with the first outlet port; and
a second device-end fitting on the second hose that mates only with the second outlet port.

11. The system as defined in claim 9 further comprising:
a key aperture disposed between the first outlet and second outlet of the positive airway pressure device; and
a bracket configured to hold the device ends of the first and second hoses in a fixed relationship, the bracket comprising a tab extending from the bracket in substantially the same direction as the device ends of the hoses;
wherein the key aperture and the tab of the bracket are configured such that the tab extends into the aperture when the hoses are fluidly coupled to the outlets; and
wherein the key aperture and the tab of the bracket allow fluid coupling of the hoses to the outlets in only one orientation.

12. The system as defined in claim 9 further comprising:
a key aperture disposed between the first outlet and second outlet of the positive airway pressure device; and
a bracket configured to hold the device ends of the first and second hoses in a fixed relationship, the bracket comprising a tab extending from the bracket in substantially the same direction as the device ends of the hoses, and the tab having features that identify the nasal mask to which the hoses are configured to attach;
wherein the key aperture and the tab of the bracket are configured such that the tab extends into the aperture when the hoses are fluidly coupled to the outlets; and
wherein the positive airway pressure device is configured to read the features of the tab that identify the nasal mask to which the noses are configured to attach.

13. The system as defined in claim 12 wherein the key aperture and the tab of the bracket allow fluid coupling of the hoses to the outlets in only one orientation.

14. The system as defined in claim 12 wherein the positive airway pressure device farther comprises:
a source of light proximate to the aperture; and
a light detector proximate to the aperture and in operational relationship to the source of light;
wherein the positive airway pressure device is configured to read the features of the tab by determining whether the tab allows light from the source of light to reach the light detector.

15. The system as defined in claim 14 further comprising:
wherein the source of light further comprises a light emitting diode; and
wherein the light detector further comprises a photo diode.

16. The system as defined in claim 12 wherein the positive airway pressure device further comprises:
a plurality of sources of light proximate to the aperture; and
a plurality of light detectors proximate to the aperture and in operational relationship one each to each source of light;
wherein the positive airway pressure device is configured to read features of the tab by determining whether the tab allows light from each source of light to reach each respective light detector.

17. The hose system as defined in claim 9 wherein the patient end of the first hose is configured to couple to a first port of a nasal mask, and wherein the patent end of the second hose is configured to couple to a second port of the nasal mask.

18. A system comprising:
a first hose having a device end and a patient end, the device end configured to couple to a first port of a positive airway pressure device, and the patient end configured to couple to a first narial port of a nasal mask;
a second hose having a device end and a patient end, the device end of the second hose configured to couple to a second port of the positive airway pressure device, and the patient end of the second hose configured to couple to a second narial port of the nasal mask; and
a means for ensuring that the device end of the first hose only couples to the first port, and the device end of the second hose only couples to the second port.

19. The system as defined in claim 18 wherein the means for ensuring further comprises:
a first device-end fitting on the first hose that mates only with the first port; and
a second device-end fitting on the second hose that mates only with the second port.

20. The system as defined in claim 18 wherein the means for ensuring further comprises:
a means for holding the device ends of the first and second hoses in a fixed relationship;
a means for orienting the means for holding such that the device end of the first hose only mates with the first port and the device end of the second hose only mates with the second port.

21. The system as defined in claim 20 further comprising a means for identifying the nasal mask to which the first and second hose are configured to attach, the means for identifying coupled to the means for holding.

22. The system as defined in claim 22 wherein the means for orienting and the means for identifying are the same device.
